(19) United States
(12) Patent Application Publication
(10) Pub. No.: US 2009/0062707 A1
(43) Pub. Date: Mar. 5, 2009

(54) Wrist support brace

(76) Inventor: Robert Busuttil, Pomona, CA (US)

Correspondence Address: Lewis M. Brande, 5976 Falling Tree Lane, Alta Loma, CA, 91737-2816, US

(21) Appl. No.: 11/899,031

(22) Filed: Sep. 4, 2007

(51) Int. Cl. A61F 5/00

(52) U.S. Cl. 602/21

(57) ABSTRACT

This invention describes a novel wrist support brace for patients with carpal tunnel syndrom or injured wrists that utilizes an adjustable strap in conjunction with a hard support brace positioned around the forearm. The brace will physically contain the injured wrist in a stable position, thereby minimizing the potential of further injury. The wrist brace will allow some movement of the wrist and permit a user to continue working while the wrist heals in a normal position.

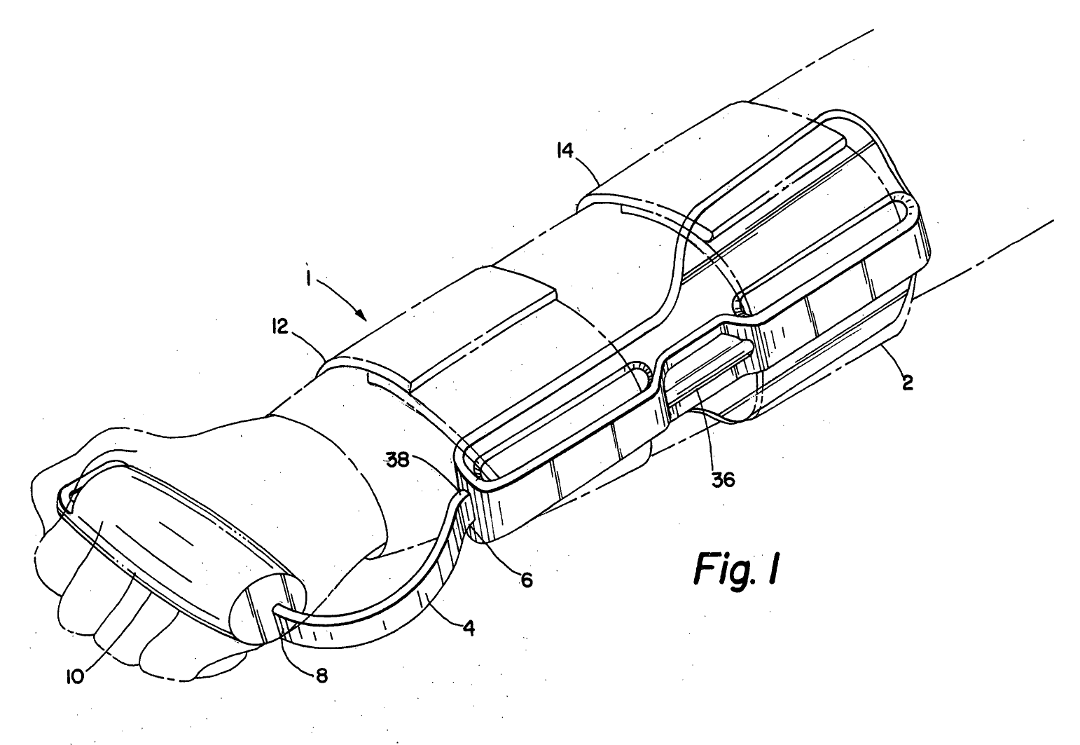

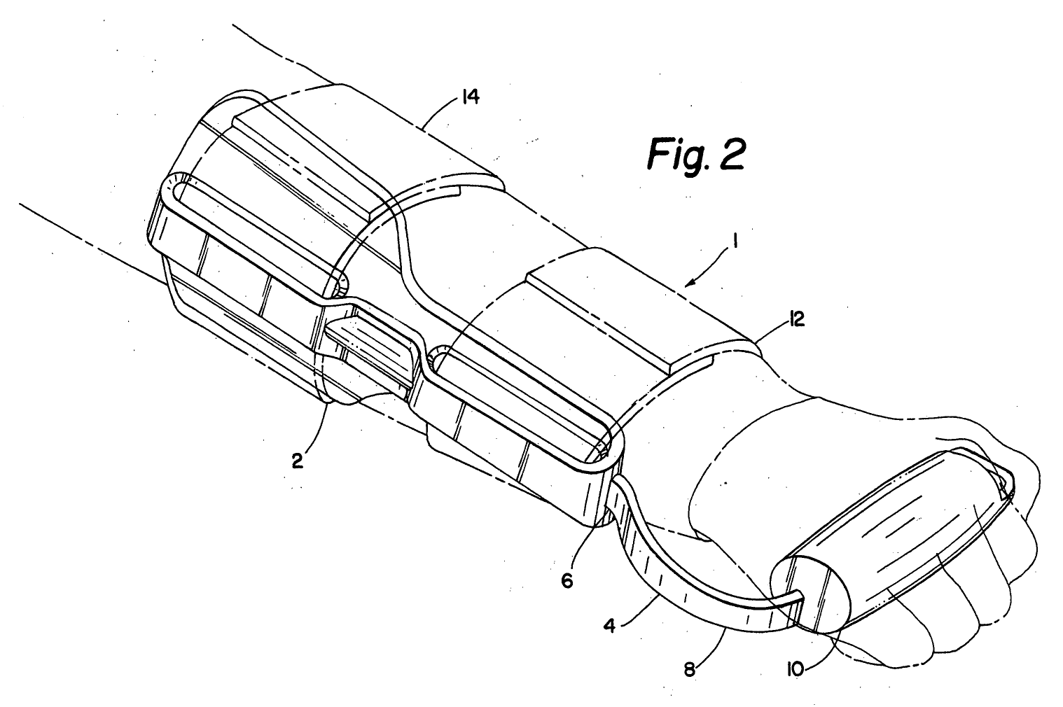

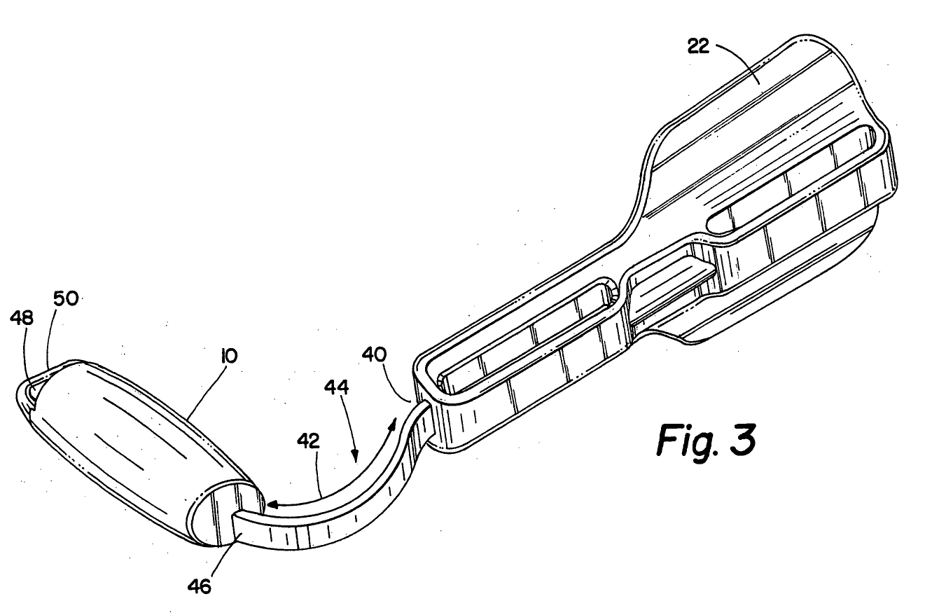

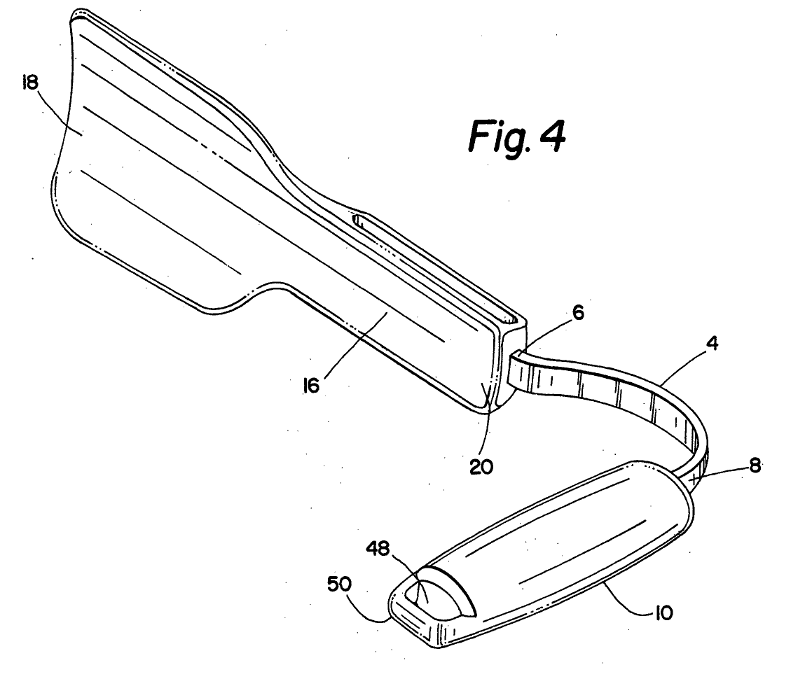

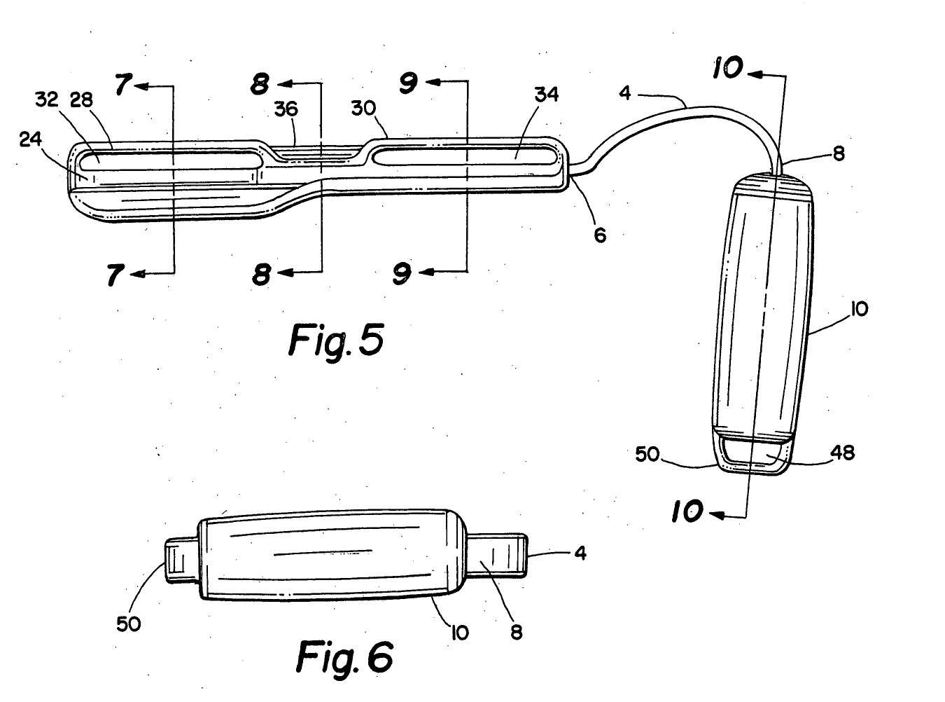

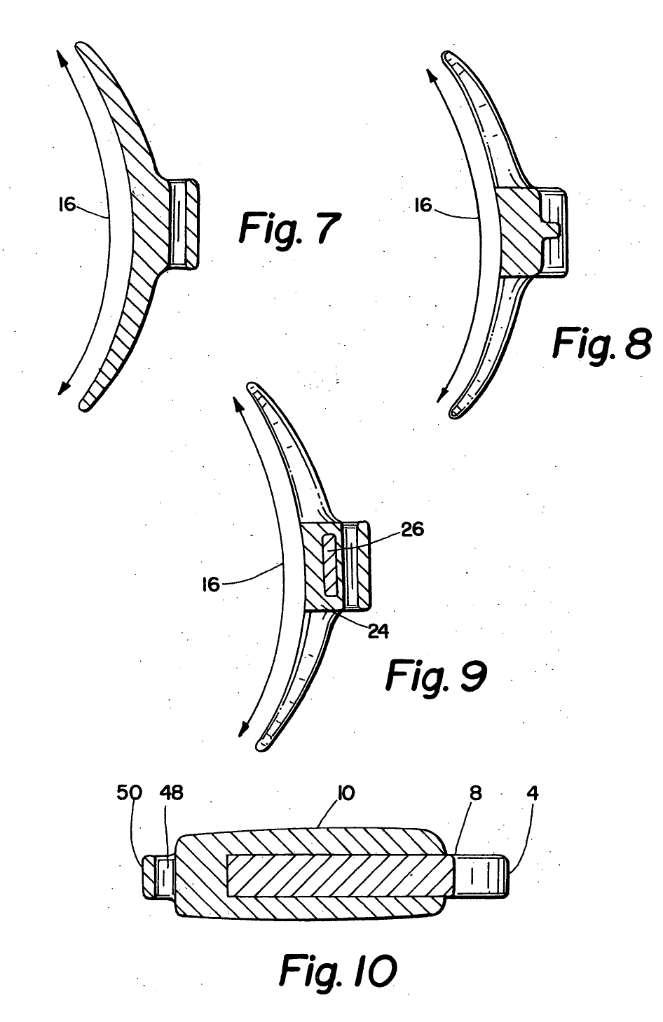

### 1. BACKGROUND OF THE INVENTION

This invention is used in combination with and comprises an improvement to U.S. Pat. No. 7,033,332 and application Ser. No. 11/728,340 and relates to the field of wrist supports. More specifically, the invention relates to an device in the field of orthotics. The device is used to assist a person who suffers from forms of strokes, paralysis, or other forms of musculature or nerve disease or damage that require manual straightening of the wrist to restore the appendage to normal everyday use.

### 2. DESCRIPTION OF THE PRIOR ART

Various types of races and appliances for therapeutic use in treating bone or muscle disorders are described in the prior art. Several patents exist to assist patients in restoring a hand that is contracted to normal condition.

A typical device is described by U.S. Pat. No. 5,437,620 by Shelly which discloses a “Wrist Splint”. This device bladders that assist in supporting the hand. The wrist is supported by rigid, nonpliable platform. Straps are used that wrap around the platform and over the wrist, applying pressure to straighten the wrist during the contractions.

Another device is U.S. Pat. No. 5,601,597 by Arrowood et al., which discloses “Combination Radial Artery Occluder and Wrist Splint”. This device is specifically used to immobilize a wrist in order to prevent further damage or injury to an artery. The device is made specifically to prevent flow through the radial artery by applying pressure and allowing blood flow through the ulnar artery. Although the device does comprise a rigid member, the rigid member is not specifically needed to straighten the wrist, but only to maintain the wrist in an immobile state.

U.S. Pat. No. 5,772,620 by Szlema et al. Discloses a “Hand and Wrist Joint Orthosis”. This device was created to immobilize an injured hand. The wrist and the fingers arc maintained in a specific orientation to each other to guarantee good recovery from an injury. The device is not designed to be used for wrist contractures, in where the wrist must be forcibly dislocated.

U.S. Pat. No. 6,106,492, publication number US 2002/0035342 A1, and US 2002/0072696 A1 are inventions that are specifically related to treatment of carpal tunnel injuries. These devices immobilize the wrist and are designed to prevent unwanted movement of the wrist, which will cause further injury to the carpal nerve system in the wrist. The devices are distinguished from the present invention in that they are not designed to apply the stabilizing force necessary to immobilize the wrist that has been stricken with an injury.

Patent Publication Number US 2002/0082538 A1 discloses an “Orthotic Device for Treating Contractures of Either Hand”. This device uses a soft padded envelope or mitt that is mated with a splint. The device has padded attachment straps that are located at different regions of the mitt. This device uses a mitt, which is made from a manually bendable material such as metal encased in a soft plastic padding. It should be noted that there is a great difference to the present invention in that the present invention is not made from bendable flat sheet metal, which is needed for Patent Publication Number US 2002/0082538 A1 to be built. Additionally the method by which the straps are used is different from the present invention, in that the straps to Patent Publication Number US 2002/0082538 A1 wrap over the wrist making it very difficult for a sole person, without assistance, to apply enough pulling force onto the straps to cause the constricted appendage to be forcibly straightened.

U.S. Pat. No. 7,033,332 by Busuttil, discloses a “Wrist Support Brace for Alleviating Contractures”, and is the parent invention from which the improvements are applied

### 3. SUMMARY OF THE INVENTION

In accordance with the present invention, there is provided a new and novel orthotic device that has been designed to be more compact than current devices and still be capable of assisting a person in stabilizing an injured wrist and alleviating mild pain, i.e. carpal tunnel syndrome of a person's wrist.

This particular device maybe used for either the left or right hand. This device has been made by bending a metallic bar that may be made from metals such as aluminum, titanium, or steel, or other materials that would provide adequate stiffness and product longevity, such as molded graphite epoxy laminates, or other plastics that may be reinforced.

The material that is used to fabricate the bar must be made stiff enough to prevent a user from bending the device out of shape. The device is comprised of one shaped bar that is positioned on the lateral portion of the appendage, i.e. the arm. The general shape would be considered a modified deep “J” shape. The bar has a straight portion, followed by a hooked, portion that is essentially perpendicular from the straight portion. The hooked portion would be fabricated to provide a comfortable shape, to allow the user to grasp it with his hand. Foam is wrapped around the hand area, to provide the user with an easily gripped surface. At least two velcro straps are included with the orthotic device, and wrap around the wrist and the forearm.

The most significant improvement over the previous inventions is making the forearm support substantially more compact than the previous inventions, allowing the device to be used for either the left or the right hand.

It is the primary object of the present invention to provide an orthotic device that can treat and stabilize carpal tunnel injuries of the wrist or hand or other injuries such as sprains or fractures of the bones. The device would be capable of immobilizing an injured arm or wrist.

A still further object of the invention is to provide an orthotic device that is easily fabricated and may be used on either wrist or hand.

Yet another object of the invention is to provide an orthotic device that has a designed shape that provides the smallest profile form, thus preventing serious interference with a patients normal use of his wrist or hand.

These and other objects, features and advantages of the present invention will be come apparent to those skilled the art from the following detailed description and attached drawings, upon which, by way of example, only a preferred and other important embodiments of the invention are described and illustrated.

### 4. BRIEF DESCRIPTION OF THE DRAWINGS

1. FIG. one (1) shows a left perspective view of the wrist support brace with a user's arm in place.

2. FIG. (2)shows a right perspective view of the wrist support brace with a user's arm in place.

3. FIG. three (3) shows a left perspective view of the wrist support brace.

4. FIG. four (4) shows a right front perspective view of the wrist support brace.

5. FIG. Five (5) shows a plan view of the wrist support brace.

6. FIG. Six (6) shows a front view of the wrist support brace.

7. FIG. Seven (7) is a cross section of the support brace at the rear most portion.

8. FIG. Eight (8) is a cross section of the support brace at a medial portion.

9. FIG. Nine (9) is a cross section of the support brace at a forward portion.

10. FIG. Ten (10) is a cross section of the support brace through the hand grip portion.

### 5. DETAILED DESCRIPTION

With respect to FIG. one (1), what is shown is a user's arm supported by a wrist support brace (1). The wrist support brace (1) is shown comprising four simple components. A forearm support (2) is shown bearing against the forearm of a user. A cross support (4) is shown having a modified “J” shape, where a first straight end (6) is shown inserted into the cross support (4). A second end (8) is shown approximately perpendicular to the first straight end (6), and provides support for a hand grip (10), where the hand grip (10) may be shaped ergonomically. A first hook and loop strap (12) and a second hook and loop strap (14) are shown placed apart and are inserted through the forearm support (2).

FIGS. five (5), seven (7), eight (8), and nine (9) allows a further breakdown of the forearm support (4) into the sub-components that further describe the novelty of the invention. A curved surface (16) is shown, where the curved surface (16) is concave shaped and bears intimately against a user's arm. The curved surface (16) of the forearm support (2) has a rear portion (18), where the rear portion (18) provides greater surface area using a greater arc length as shown in FIG. seven (7), for maximum support and comfort when bearing against a user's forearm. The curved surface (16) of the forearm support (2) has a forward portion (20) where the forward portion (20) has less surface area because the forward portion (20) uses a smaller sector of arc length than the rear portion (18), but is carried over a longer linear distance. Centrally positioned on an outer portion (22) of the curved surface (16) of the forearm support (2) is a first boss (24) where the first boss (24) is shown running the full length of the forearm support (2). The first boss (24) has a centrally defined hollow area (26) where the hollow area (26) is designed to provide a tight friction fit with the cross support (4) preventing any undesired slip between the forearm support (2) and the cross support (4). The hollow area (26) may run the full length of the forearm support (4).

The wrist support brace (1) is further shown having a second boss (28) and a third boss (30). The second boss (28) is biased towards the rear of the wrist support brace (1), while the third boss (30) is biased towards the front of the wrist support brace (1). In the present embodiment, the second boss (28) and the third boss (30) are shown placed on an outward side (52) of said first boss(24). The second boss (28) has a first slot (32) defined therein, while the third boss (30) has a second slot (34) defined therein. The first slot (32) and the second slot (34) are shown having a width wide enough to allow a sliding fit for the first hook and loop strap (12), and second hook and loop strap (14). The first and second slots (32, 34) additionally have smooth edges to prevent chafing against the first hook and loop strap (12), and second hook and loop strap (14). A stiffening bar (36) is shown between the second and third bosses (28, 30) and provides additional bending stiffness to the wrist support brace (1).

The cross support (4) is shown having a modified “J” shape, and is described in detail as follows. The cross support (4) has a first straight portion (38), where the first straight portion (38) is shown inserted through the hollow area (26) of the first boss (24). The cross support (4) is then shown having a reverse curve portion (40) that curves away from the forearm support (2). The cross support (4) is shown having a smooth return curve portion (42) that curves towards the forearm support (4) and creates a clearance cavity (44). The return curve portion (42) is shown becoming essentially perpendicular to the first straight portion (3 8), creating a second straight portion (46) that provides a good interface with the hand grip (10). The second straight portion (46) may have a slight bend or curve to create a tighter fit with the hand grip (10).

The hand grip (10) of the wrist support brace (1) is shown inserted onto the second straight end (8). The hand grip (10) may have differing shapes, the most common being a surface of revolution that is easy to fabricate. Another shape would be more oblong in cross section, which would provide the user with a more comfortable fit. The hand grip (10) may also be shaped ergonomically, which is a shape that is defined by specialists for the best comfort and support. The hand grip (10) may be made from materials such as foams and plastics or a combination of materials that is defined by specialists for the specific condition being treated. The hand grip (10) is shown having a loop (48), where the loop is defined at the terminus free end (50) of the hand grip (10).

Although the foregoing includes a description of the best mode contemplated for carrying out the invention, various modifications are contemplated.

As various modifications could be made in the constructions herein described and illustrated without departing from the scope of the invention, it is intended that all matter contained in the foregoing description or shown in the accompanying drawings shall be interpreted as illustrative rather than limiting.

What is claimed is:

1. A wrist support brace comprising:
a. a forearm support, said forearm support has a front portion and a rear portion, said rear portion is curve shaped, said front portion also is curved shaped, said rear portion has a greater arc length than said front portion, said rear portion providing a greater surface area than said front portion;
b. a first boss, said first boss is centrally located on an outer portion of said curved surface, said first boss running the full length of said forearm support;
c. said first boss has a centrally defined hollow area defined therein, said centrally defined hollow area providing a tight friction fit for a cross support;
d. said forearm support further has a second boss and a third boss defined therein, said second boss is rearwardly biased, said third boss is forwardly biased on said forearm support, said first and said second bosses is placed on an outward side of said first boss, said second boss has a first slot defined therein, said third boss has a second slot defined therein, said first slot and said second slot each has a allowing a sliding fit for a first hook and loop strap, and a second hook and loop strap said first and second slots additionally have smooth edges preventing chafing against said first hook and loop strap, and said second hook and loop strap;
e. a stiffening bar is shown positioned between said second and said third bosses providing additional bending stiffness to the wrist support brace thereby;
f. a cross support, said cross support has a first straight portion, said first straight portion is inserted through said hollow area of said first boss, said cross support further has a reverse curve portion, said reverse curve portion curving away from said forearm support, said cross support additionally has a smooth return curve portion, said smooth return curve portion curving towards said forearm support creating a clearance cavity thereby, said return curve portion is placed perpendicular to said first straight portion, creating a second straight portion providing a good interface with a hand grip thereby; and
g. said hand grip is inserted onto said second straight end of said cross support.

2. A wrist support brace comprising:
a. a forearm support, said forearm support has a first boss attached thereon, said first boss has a centrally positioned hollow area theredefined;
b. a cross support, said cross support is inserted into said centrally positioned hollow portion of said first boss, said cross support is frictionally contained thereby, said cross support has a modified “J” shape;
c. said forearm support has a second boss and a third boss, said second and said third boss is attached onto said first boss, said second boss is rearwardly biased, said third boss is forwardly biased;
d. said second boss has a first slot theredefined, said third boss has a second slot theredefined, said first slot and said second slot allow a first strap means and a second strap means to be inserted respectively thereby; and
e. a hand grip, said hand grip is attached onto said cross support.

3. The wrist support brace of claim 2, wherein said forearm support has a curved shape, said curved shape is concave towards a users forearm and wrist.

4. The wrist support brace of claim 2, wherein said cross support further has a first straight portion, said first straight portion is inserted through said hollow area of said first boss, said cross support further has a reverse curve portion, said reverse curve portion curving away from said forearm support, said cross support additionally has a smooth return curve portion, said smooth return curve portion curving towards said forearm support creating a clearance cavity thereby, said return curve portion is placed perpendicular to said first straight portion, creating a second straight portion providing a good interface with said hand grip.

5. The wrist support brace of claim 2, further comprising a stiffening bar, said stiffening bar is positioned between said second and said third bosses providing additional bending stiffness to the wrist support brace thereby.

6. The wrist support brace of claim 2, wherein;
a. said first strap means is a hook and loop strap and
b. said second strap means is a hook and loop strap.

7. The wrist support brace of claim 2, wherein said hand grip is ergonomically shaped.

8. The wrist support brace of claim 7 wherein said hand grip has a loop, said loop is defined at a terminus free end of said hand grip.
